(19) United States
(12) Patent Application Publication
(10) Pub. No.: US 2007/0197610 A1
(43) Pub. Date: Aug. 23, 2007

(54) Benzisoxazoles

(75) Inventors: Ludo Edmond Josephine Kennis, Lier (BE); Greta Constantia Peter Vanhoof, Zoersel (BE); Jean-Pierre Andre Marc Bongartz, Turnhout (BE); Marcel Gerebernus Maria Luyckx, Geel (BE); Wenda Eveline Minke, Breda (NL)

Correspondence Address: WOODCOCK WASHBURN LLP, CIRA CENTRE, 12TH FLOOR, 2929 ARCH STREET, PHILADELPHIA, PA, 19104-2891, US

(73) Assignee: Janssen Pharmaceutica N.V.

(21) Appl. No.: 10/598,957

(22) PCT Filed: Mar. 11, 2005
(86) PCT No.: PCT/EP05/51105

§ 371 Date: Sep. 15, 2006

Related U.S. Application Data
(60) Provisional application No. 60/554,231, filed on Mar. 18, 2004.

(30) Foreign Application Priority Data
Mar. 16, 2004 (EP) 04101068.7

(51) Int. Cl. A61K 31/426; A61K 31/428; A61K 31/425; A61K 31/42; C07D 413/02; C07D 417/02

(52) U.S. Cl. 514/365; 514/373; 514/379; 514/367; 548/152; 548/181; 546/216; 548/235; 548/241; 548/207

(57) ABSTRACT

The present invention concerns the compounds of formula (I), the N-oxide forms, the pharmaceutically acceptable addition salts and the stereochemically isomeric forms thereof, wherein m represents an integer from 1 to 3; X represents amino, hydroxy, -oxo or -Z-R¹; Y is absent when X represents -Z-R¹ and —(C═O)—R⁶ when X represents oxo; Z represents carbonyl, -oxy-carbonyl- or —NR⁵-carbonyl-; R¹ represents C₁₋₄alkyl, Ar¹, Ar¹—C₁₋₄alkyl-, —NR³R⁴ or -Het¹; R² represents hydrogen, halo, nitro, hydroxycarbonyl-, C₁₋₄alkyloxy or C₁₋₄alkyl; R³ and R⁴ are each independently selected from hydrogen, Ar³ or C₁₋₄alkyl; R⁵ represents hydrogen, C₁₋₄alkylcarbonyl- or Ar⁴-carbonyl-; R⁶ represents a substituent selected from the group consisting of C₁₋₄alkyl, Ar⁵, Ar⁶—C₁₋₄alkyl- or NR⁷R⁸; R⁷ and R⁸ are each independently selected from hydrogen, Het⁴ or C₁₋₄alkyl; Het¹ represents a heterocycle selected from oxazolyl, isoxazolyl, imidazolyl or pyrazolyl wherein said heterocycle is optionally substituted with one, two or three substitutents selected from the group consisting of amino, C₁₋₄alkyl, hydroxy-C₁₋₄alkyl, phenyl, phenyl-C₁₋₄alkyl- and phenyl substituted with one or more halo substitutents; Het⁴ represents a heterocycle selected from oxazolyl or isoxazolyl, wherein said heterocycle is optionally substituted with one or more substitutents selected from the group consisting of amino, C₁₋₄alkyl, hydroxy-C₁₋₄alkyl-, phenyl, phenyl-C₁₋₄alkyl and phenyl substituted with one or more halo substitutents; and Ar¹, Ar², Ar³, Ar⁴, Ar⁵ or Ar⁶ each independently represents phenyl optionally substituted one or where possible two or more substitutents selected from halo, nitro, C₁₋₄alkyl, hydroxy or C₁₋₄alkyloxy-.

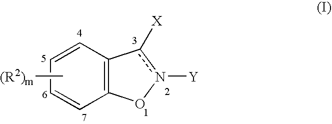

This invention relates to benzisoxazole derivatives exhibiting D-amino oxidase inhibitory activity and therapeutic effects or preventive effects on mental disorders including schizophrenia.

D-Amino acid oxidase (DAAO) is a highly selective enzyme that degrades in particular, neutral D-amino acids with a preference for those having small side chains, in particular D-serine and D-alanine. It could act as a detoxifying agent which removes D-amino acids derived from endogenous and exogenous sources but was more recently identified as a gene involved in the regulation of the N-methyl-D-aspartate (NMDA) receptor pathway in schizophrenia (Chumakov I., et al., PNAS (2002) Vol. 99, pp. 13675-13680).

Exacerbation of psychotic symptoms in schizophrenic patients and psychotomimetic effects in normal humans following administration of phencyclidine or ketamine, both NMDA receptor antagonists, indicate the involvement of NMDA receptor dysfunction in the pathophysiology of schizophrenia.

Functional activation of NMDA receptors through glutamate requires that the modulatory site of this ligand-gated ion channel is occupied by a co-agonist. D-serine is a potent endogenous co-agonist of the strychnine insensitive glycine site of the NMDA receptor (Mothet J-P., et al., PNAS (2000) Vol. 97, pp. 4926-4931). The activity of DAAO selectively depletes D-serine in the brain and accordingly can attenuate NMDA-type glutamate receptor activity, which could result in glutamate signalling hypofunction, a mechanism recently proposed in schizophrenia. It is thus an object of the invention to provide compounds that selectively reduce DAAO activity and hence ameliorate the impaired NMDA-type glutamate receptor activity in schizophrenia patients.

It is known, see for example PCT International Patent Publication WO 93/16073 and European Patent Application EP 353 821 that 3-piperazinyl and 3-piperidinyl-benzisoxazoles have an anti-psychotic activity and are useful as anxiolytics, muscle relaxants, antidepressants, antiemetics, and in the treatment of aggression associated with senile dementia as well as in the treatment of personality disorders including schizophrenia. These compounds address the dopaminergic pathway in mental disorders and accordingly treat the positive symptoms, i.e. hallucinations and delusions, in schizophrenic patients. In PCT International Patent Publication WO 94/12495 it was shown that certain 3-(aminoalkylamino)-1,2-benzisoxazoles and related compounds are useful for the treatment of various memory dysfunctions characterized by a decreased cholinergic function such as Alzheimer's disease. Some of the compounds described in WO 94/12495 were also found to inhibit monoamine oxidase and hence are useful as antidepressants. A similar utility is associated to the substituted (pyridinylamino)-benzisoxazoles disclosed in the European patent application EP 594 000.

Compared to the 3-piperazinyl- and 3-piperidinyl-benzisoxazoles, the particular structural differences of the compounds of the present invention, make these compounds DAAO antagonists. As such these compounds address the GABAergic pathway that is associated with the negative symptoms, i.e. impoverishment of affect, thought, and initiative, or other cognitive disturbances of schizophrenia.

Further benzisoxazoles comprising a 3-alkyloxy-amino or di (C₁₋₄alkyl)amino substitutent are described in European Patent Application EP 779 281 and Japanese Patent Applications JP Sho 52-031070 and JP Sho 57-021377 as local anaesthetics, antihistaminic agents, anti-inflammatory agents, as having cardiovascular effects, in particular as β-blockers and to have therapeutic and preventive effects on neuropathies including Parkinson's disease, depression and Alzheimer's disease. It is however, fully unknown that these compounds have DAAO inhibitory activity and are accordingly useful for the treatment of mental disorders, such as in particular schizophrenia.

It is accordingly a first object of the present invention to provide the use of benzisoxazole derivatives of formula (I) in the manufacture of a medicament for treatment of mental disorders, in particular schizophrenia and other diseases linked to NMDA receptor dysfunction including pain, spasticity, epilepsy, and diseases with impaired learning and memory such as Alzheimer's disease, Huntington's disease, Parkinson's disease, AIDS, Attention Deficit Disorder, Attention Deficit Hyperactivity Disorder, and autism.

The benzisoxazole derivatives of formula (I) as used hereinbefore, consist of the compounds of formula (I) 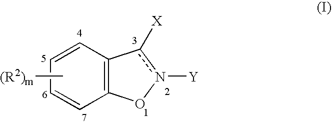
the N-oxide forms, the pharmaceutically acceptable addition salts and the stereochemically isomeric forms thereof, wherein
m represents an integer from 1 to 3;
X represents hydroxy, amino, oxo or -Z-R¹;
Y is absent or represents —C═O)—R⁶;
Z represents carbonyl, -oxy-carbonyl-, ═N-carbonyl- or —NR⁵-carbonyl;
R¹ represents hydrogen, C₁₋₄alkyl, C₁₋₄alkyloxy-, Ar¹, Ar²—C₁₋₄alkyl-, —NR³R⁴ or -Het¹;
R² represents hydrogen, halo, hydroxy, nitro, cyano, hydroxycarbonyl-, amino, mono- or di (C₁₋₄alkyl)amino-, C₁₋₆alkyloxycarbonyl-, C₁₋₄alkyloxycarbonylC₁₋₄alkyloxy-, C₁₋₄alkyloxy- optionally substituted with one or more halo atoms or R² represents C₁₋₄alkyl optionally substituted with one or more halogen atoms;
R³ and R⁴ are each independently selected from hydrogen, Het², Ar³, C₁₋₄alkyl or C₁₋₄alkyl substituted with one or more substitutents selected from halo, hydroxy or C₁₋₄alkyloxy-;
R⁵ represents hydrogen, C₁₋₄alkyl, C₁₋₄alkylcarbonyl, C₁₋₄alkyloxycarbonyl- or Ar⁴-carbonyl-;
R⁶ represents a substitutent selected from the group consisting of C₁₋₄alkyl, C₁₋₄alkyloxy-, Ar⁵, Ar⁶—C₁₋₄alkyl-, —NR⁷R⁸ or Het³;
R⁷ and R¹ are each independently selected from hydrogen, Het⁴, Ar⁷, C₁₋₄alkyl or C₁₋₄alkyl substituted with one or more substitutents selected from halo, hydroxy or C₁₋₄alkyloxy-;
Het¹ represents a heterocycle selected from oxazolyl, isoxazolyl, thiazolyl, isothiazolyl, imidazolyl, pyrazolyl, benzisoxazolyl, benzimidazolyl or benzothiazolyl wherein said heterocycle is optionally substituted with one or more substitutents each independently selected from the group consisting of amino, C₁₋₄alkyl, hydroxy-C₁₋₄alkyl-, phenyl, phenyl-C₁₋₄alkyl- and phenyl substituted with one or more halo substitutents;
Het² represents a heterocycle selected from oxazolyl, isoxazolyl, thiazolyl, isothiazolyl, imidazolyl, pyrazolyl, benzisoxazolyl, benzimidazolyl or benzothiazolyl wherein said heterocycle is optionally substituted with one or more substitutents each independently selected from the group consisting of amino, C₁₋₄alkyl, hydroxy-C₁₋₄alkyl-, phenyl, phenyl-C₁₋₄alkyl- and phenyl substituted with one or more halo substitutents;
Het³ represents a heterocycle selected from oxazolyl, isoxazolyl, thiazolyl, isothiazolyl, imidazolyl, pyrazolyl, benzisoxazolyl, benzimidazolyl or benzothiazolyl wherein said heterocycle is optionally substituted with one or more substitutents each independently selected from the group consisting of amino, C₁₋₄alkyl, hydroxy-C₁₋₄alkyl-, phenyl, phenyl-C₁₋₄alkyl- and phenyl substituted with one or more halo substitutents;
Het⁴ represents a heterocycle selected from oxazolyl, isoxazolyl, thiazolyl, isothiazolyl, imidazolyl, pyrazolyl, benzisoxazolyl, benzimidazolyl or benzothiazolyl wherein said heterocycle is optionally substituted with one or more substitutents each independently selected from the group consisting of amino, C₁₋₄alkyl, hydroxy-C₁₋₄alkyl-, phenyl, phenyl-C₁₋₄alkyl- and phenyl substituted with one or more halo substitutents;
Ar¹, Ar², Ar³, Ar⁴, Ar⁵, Ar⁶ or Ar⁷ each independently represents phenyl optionally substituted one or where possible two or more substitutents selected from halo, nitro, C₁₋₄alkyl, hydroxy or C₁₋₄alkyloxy-.

In a further object of this invention it was found that the intermediate products in the synthesis of the compounds of formula (I), i.e. the 3-amino-1,2-benzisoxazole derivatives and 3-hydroxy-1,2-benzisoxazole derivatives thereof (compounds of formula (Ig) and (Ia) hereinafter), have DAAO inhibiting activity. In WO 00/027199 it was demonstrated that some substituted 3-amino-1,2-benzisoxazole derivatives have anti-thrombin activity and are accordingly useful in the treatment of thromboembolic diseases, general hypercoagulable states or local hypercoagulable states, such as following angioplasty and coronary bypass operations. Similarly, for some of the 3-hydroxy-1,2-benzisoxazole derivatives it is known that they have morphogenetic and cell elongating activity (Branca C., et al., Plant Cell Reports (1991), 10(10), 498-500), but it was hitherto unknown that these derivatives as well as the 3-amino-1,2-benzisoxazole derivatives have DAAO inhibitory activity and are accordingly useful in the treatment of mental disorders as described hereinbefore.

It is accordingly an object of the present invention to provide the use of compounds of formula (Ia) or (Ig) in the manufacture of a medicament for treatment of mental disorders, such as for example schizophrenia, 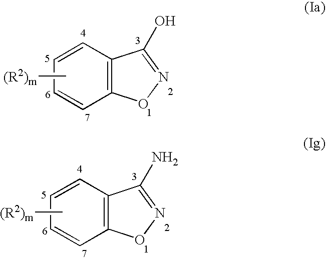
the N-oxide forms, the pharmaceutically acceptable addition salts and the stereochemically isomeric forms thereof, wherein
m represents an integer from 1 to 3; in particular m represents 1;
R² represents hydrogen, halo, hydroxy, nitro, cyano, hydroxycarbonyl-, amino, mono- or di(C₁₋₄alkyl)amino-, C₁₋₆alkyloxycarbonyl-, C₁₋₄alkyloxycarbonylC₁₋₄alkyloxy-, C₁₋₄alkyloxy- optionally substituted with one or more halo atoms or R² represents C₁₋₄alkyl optionally substituted with one or more halogen atoms; in another embodiment of the present invention R² represents a substitutent selected from the group consisting of hydrogen, halo, nitro, hydroxycarbomyl-, C₁₋₄alkyloxy- or C₁₋₄alkyl; in a further embodiment of the present invention R² represents a substitutent selected from the group consisting of hydrogen, halo, nitro, hydroxycarbomyl-, C₁₋₄alkyloxy-, C₁₋₄alkyl or C₁₋₄alkyl substituted with one or more halo atoms; in an even further embodiment of the present invention R² represents hydrogen, chloro, nitro, methyl, methoxy or hydroxycarbonyl; in a particular embodiment R² represents hydrogen, chloro, fluoro, bromo, iodo, trifluoromethyl, nitro, methyl, methoxy or hydroxycarbonyl.

It is accordingly an object of this invention to provide the use of an intermediate of formula (Ia) in the manufacture of a medicament for treating the impaired NMDA-type glutamate receptor activity in schizophrenia patients and other diseases linked to NMDA receptor dysfunction including pain, spasticity, epilepsy, and diseases with impaired learning and memory such as Alzheimer's disease, Huntington's disease, Parkinson's disease, AIDS, attention deficit disorder, attention deficit hyperactivity disorder, and autism.

In addition, for those compounds of formula (I) wherein X represents -Z-R¹ it was found that they tend to hydrolyse into the 3-amino or 3-hydroxy derivatives of formula (Ig) and (Ia), without loss of DAAO inhibitory activity. Hence, in a further object of this invention, the compounds of formula (I) wherein X represents -Z-R¹ are useful as prodrugs in the treatment of mental disorders as described hereinbefore, since when administered to a biological system, said compounds are converted into further biologically active compounds as a result of spontaneous chemical reaction(s), enzyme catalysed chemical reaction(s) and/or metabolic chemical reaction(s), or a combination of each. Notwithstanding the fact that the compounds of formula (I) wherein X represents -Z-R¹ have DAAO inhibitory activity, this activity is typically less than the activity of the 3-amino or 3-hydroxy derivatives. Hence the use of said compounds as prodrugs serves to improve drug efficacy or safety through improved oral bioavailability, pharmacodynamic half-life, etc.

As used in the foregoing definitions and hereinafter,

halo is generic to fluoro, chloro, bromo and iodo;

C₁₋₄alkyl defines straight and branched chain saturated hydrocarbon radicals having from 1 to 4 carbon atoms such as, for example, methyl, ethyl, propyl, butyl, 1-methylethyl, 2-methylpropyl, 2,2-dimethylethyl and the like;

C₁₋₆alkyl is meant to include C₁₋₄alkyl and the higher homologues thereof having 6 carbon atoms such as, for example hexyl, 1,2-dimethylbutyl, 2-methylpentyl and the like;

C₁₋₄alkyloxy defines straight or branched saturated hydrocarbon radicals having from 1 to 4 carbon atoms and 1 oxygen atom such as methoxy, ethoxy, propyloxy, butyloxy, 1-methylethyloxy, 2-methylpropyloxy and the like;

carbonyl (i), oxy-carbonyl- (ii), ═N-carbonyl- (iii) and NR⁵-carbonyl (iv) define bivalent radicals of the following formula; 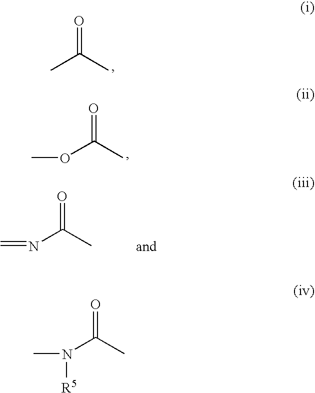
respectively

oxo defines an oxygen atom that taken together with the carbon atom to which it is attached forms a carbonyl moiety.

The heterocycles as mentioned in the above definitions and hereinafter, are meant to include all possible isomeric forms thereof, for instance triazolyl also includes 1,2,4-triazolyl and 1,3,4-triazolyl; oxadiazolyl includes 1,2,3-oxadiazolyl, 1,2,4-oxadiazolyl, 1,2,5-oxadiazolyl and 1,3,4-oxadiazolyl; thiadiazolyl includes 1,2,3-thiadiazolyl, 1,2,4-thiadiazolyl, 1,2,5-thiadiazolyl and 1,3,4-thiadiazolyl.

Further, the heterocycles as mentioned in the above definitions and hereinafter may be attached to the remainder of the molecule of formula (I) through any ring carbon or heteroatom as appropriate. Thus, for example, when the heterocycle is imidazolyl, it may be a 1-imidazolyl, 2-imidazolyl, 3-imidazolyl, 4-imidazolyl and 5-imidazolyl; when it is thiazolyl, it may be 2-thiazolyl, 4-thiazolyl and 5-thiazolyl; when it is benzothiazolyl, it may be 2-benzothiazolyl, 4-benzothiazolyl, 5-benzothiazolyl, 6-benzothiazolyl and 7-benzothiazolyl.

The pharmaceutically acceptable addition salts as mentioned hereinabove are meant to comprise the therapeutically active non-toxic acid addition salt forms which the compounds of formula (I) are able to form. The latter can conveniently be obtained by treating the base form with such appropriate acid. Appropriate acids comprise, for example, inorganic acids such as hydrohalic acids, e.g. hydrochloric or hydrobromic acid; sulfuric; nitric; phosphoric and the like acids; or organic acids such as, for example, acetic, propanoic, hydroxyacetic, lacetic, pyruvic, oxalic, malonic, succinic (i.e. butanedioic acid), maleic, fumaric, malic, tartaric, citric, methanesulfonic, ethanesulfonic, benzenesulfonic, p-toluenesulfonic, cyclamic, salicylic, p-aminosalicylic, pamoic and the like acids.

The pharmaceutically acceptable addition salts as mentioned hereinabove are meant to comprise the therapeutically active non-toxic base addition salt forms which the compounds of formula (I) are able to form. Examples of such base addition salt forms are, for example, the sodium, potassium, calcium salts, and also the salts with pharmaceutically acceptable amines such as, for example, ammonia, alkylamines, benzathine, N-methyl-D-glucamine, hydrabamine, amino acids, e.g. arginine, lysine.

Conversely said salt forms can be converted by treatment with an appropriate base or acid into the free acid or base form.

The term addition salt as used hereinabove also comprises the solvates which the compounds of formula (I) as well as the salts thereof, are able to form. Such solvates are for example hydrates, alcoholates and the like.

The term stereochemically isomeric forms as used hereinbefore defines the possible different isomeric as well as conformational forms which the compounds of formula (I) may possess. Unless otherwise mentioned or indicated, the chemical designation of compounds denotes the mixture of all possible stereochemically and conformationally isomeric forms, said mixtures containing all diastereomers, enantiomers and/or conformers of the basic molecular structure. All stereochemically isomeric forms of the compounds of formula (I) both in pure form or in admixture with each other are intended to be embraced within the scope of the present invention.

The N-oxide forms of the compounds of formula (I) are meant to comprise those compounds of formula (I) wherein one or several nitrogen atoms are oxidized to the so-called N-oxide, particularly those N-oxides wherein the benzisoxazole-nitrogen is N-oxidized.

In a particular embodiment the present invention provides the use of compounds of formula (I) wherein one or more of the following restrictions apply;
m represents an integer from 1 to 3;
X represents -oxo or -Z-R¹;
Y is absent when X represents -Z-R¹ and —(C═O)—R⁶ when X represents oxo;
Z represents carbonyl, -oxy-carbonyl- or —NR⁵-carbonyl-;
R¹ represents C₁₋₄alkyl, Ar¹, Ar¹—C₁₋₄alkyl-, —NR³R⁴ or -Het¹;
R² represents hydrogen, halo, nitro, hydroxycarbonyl-, C₁₋₄alkyloxy or C₁₋₄alkyl optionally substituted with one or more halo atoms; in particular R² represents hydrogen, halo, nitro, hydroxycarbonyl-, C₁₋₄alkyloxy or C₁₋₄alkyl;
R³ and R⁴ are each independently selected from hydrogen, Ar³ or C₁₋₄alkyl;
R⁵ represents hydrogen, C₁₋₄alkylcarbonyl- or Ar⁴-carbonyl-;
R⁶ represents a substitutent selected from the group consisting of C₁₋₄alkyl, Ar⁵, Ar⁶—C₁₋₄alkyl- or NR⁷R⁸;
R⁷ and R⁸ are each independently selected from hydrogen, Het⁴ or C₁₋₄alkyl;
Het¹ represents a heterocycle selected from oxazolyl, isoxazolyl, imidazolyl or pyrazolyl wherein said heterocycle is optionally substituted with one, two or three substitutents selected from the group consisting of amino, C₁₋₄alkyl, hydroxy-C₁₋₄alkyl, phenyl, phenyl-C₁₋₄alkyl- and phenyl substituted with one or more halo substitutents, in particular said heterocycle is substituted with one or more substitutents selected from the group consisting of C₁₋₄alkyl, phenyl or phenyl substituted with one or more halo substitutents; in a particular embodiment Het¹ represents a heterocycle selected from isoxazolyl and pyrazolyl wherein said heterocycle is substituted with one or more substitutents selected from the group consisting of amino, C₁₋₄alkyl, hydroxy-C₁₋₄alkyl, phenyl, phenyl-C₁₋₄alkyl- and phenyl substituted with one or more halo substitutents, in particular said heterocycle is substituted with one or more substitutents selected from the group consisting of C₁₋₄alkyl, phenyl or phenyl substituted with one or more halo substitutents;
Het⁴ represents a heterocycle selected from oxazolyl or isoxazolyl, wherein said heterocycle is optionally substituted with one or more substitutents selected from the group consisting of amino, C₁₋₄alkyl, hydroxy-C₁₋₄alkyl-, phenyl, phenyl-C₁₋₄alkyl and phenyl substituted with one or more halo substitutents, in particular said heterocycle is substituted with one or more substitutents selected from C₁₋₄alkyl, phenyl or phenyl substituted with one or more halo substitutents; in a particular embodiment Het⁴ represents isoxazolyl substituted with one or more substitutents selected from C₁₋₄alkyl, phenyl or phenyl substituted with one or more halo substitutents;
Ar¹, Ar², Ar³, Ar⁴, Ar⁵ or Ar⁶ each independently represents phenyl;
   in the manufacture of a medicament for the treatment of mental disorders as defined hereinbefore.

It is a further objective of the present invention to provide novel compounds with DAAO inhibiting activity, said compounds having the formula (I) as defined hereinbefore provided however that when;
Z is -oxycarbonyl and R¹ is chloro- or nitro-phenyl-, then R² is not methyloxy-, ethyloxy-, chloro or fluoro,
Z is -oxycarbonyl and R¹ is methyl, methyloxy-, ethyloxy-, phenyl, chlorophenyl, nitrophenyl, isoxazolyl substituted with chloro or methyl or when R¹ is pyrazolyl substituted with ethyl and methyl, then R² is not hydrogen, chloro, fluoro, bromo, ethyloxy, methyloxy or methyl,
Z is —NR⁵-carbonyl and R¹ is methyl, methyloxy-, ethyloxy-, t-butyloxy-, benzyloxy-, phenyl or di-chlorophenyl, then R² is not hydrogen, halo, methyl or trifluoromethyl,
Z is oxycarbonyl and R³ or R⁴ is a methyl, isopropyl, propyl, t-butyl or an isoxazolyl substituted with either chloro, one methyl substitutent or with one methyl and one di-chloro-phenyl substitutent, then R² is not hydrogen, chloro or methyl.

With the aforementioned provisos;
the particular benzisoxazoles available in the Maybridge plc HS catalog,
the particular 1,2-benzisoxazoles disclosed in Science of Synthesis (2002), 11, p. 289-335,
the particular polyamides obtained from active diacyl derivatives of 3-hydroxy-1,2-benzisoxazoles as disclosed in Journal of Polymer Science (1981), 19(5), p. 1061-1071,
the acyl derivatives of 3-hydroxy-1,2-benzisoxazoles as disclosed in Acta Poloniae Pharmaceutica (1984), 41(6), p. 625-631; Polish Journal of Pharmacology and Pharmacy (1978), 30(5), p. 1061-1071; Polish Journal of Thermal Analysis (1979), 15(2), p. 257-260 and Chemische Berichte (1969), 102(11), p. 3775-3785,
the particular 3-substituted 1,2-benzisoxazoles disclosed in Japanese patent application JP 80-95447, and
the particular 3-acylaminobenzisoxazoles disclosed in Journal of Heterocyclic Chemistry (1973), 10(6), p. 957-961

are excluded from the present class of novel DAAO inhibitors.

An interesting group of compounds are those compounds of formula (I) wherein Z represents oxy-carbonyl or NR⁵-carbonyl, hereinafter referred to as the compounds of formula (Ic) 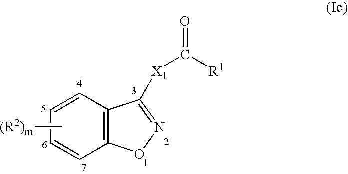
the N-oxide forms, the pharmaceutically acceptable addition salts and the stereochemically isomeric forms thereof, wherein
m represents an integer from 1 to 3;
X₁ represents O or NR⁵;
R¹ represents C₁₋₄alkyl, C₁₋₄alkyloxy-, —Ar¹, Ar²—C₁₋₄alkyl-, —NR³R⁴ or Het¹;
R² represents hydrogen, halo, hydroxy, nitro, hydroxycarbonyl-, amino, mono- or di (C₁₋₄alkyl)amino, C₁₋₆alkyloxycarbonyl-, C₁₋₄alkyloxycarbonylC₁₋₄alkyloxy-, C₁₋₄alkyloxy- optionally substituted with one or more halo atoms or R² represents C₁₋₄alkyl optionally substituted with one or more halogen atoms;
R³ and R⁴ are each independently selected from hydrogen, Het², phenyl, C₁₋₄alkyl or C₁₋₄alkyl substituted with one or more substitutents selected from halo, hydroxyl, phenyl or C₁₋₄alkyloxy-;
R⁵ represents hydrogen, C₁₋₄alkyl, phenyl-carbonyl- or C₁₋₄alkyl-carbonyl-;
Het¹ represents a heterocycle selected from oxazolyl, isoxazolyl, thiazolyl, isothiazolyl, imidazolyl, pyrazolyl, benzisoxazolyl, benzimidazolyl or benzothiazolyl wherein said heterocycle is optionally substituted with one or more substitutents each independently selected from the group consisting of amino, C₁₋₄alkyl, hydroxy-C₁₋₄alkyl-, phenyl, phenyl-C₁₋₄alkyl- and phenyl substituted with one or more halo substitutents;
Het² represents a heterocycle selected from oxazolyl, isoxazolyl, thiazolyl, isothiazolyl, imidazolyl, pyrazolyl, benzisoxazolyl, benzimidazolyl or benzothiazolyl wherein said heterocycle is optionally substituted with one or more substitutents each independently selected from the group consisting of amino, C₁₋₄alkyl, hydroxy-C₁₋₄alkyl-, phenyl, phenyl-C₁₋₄alkyl- and phenyl substituted with one or more halo substitutents,
provided that when;
X₁ is —O— and R¹ is methyl, methyloxy-, ethyloxy-, phenyl, chlorophenyl, nitrophenyl, isoxazolyl substituted with chloro or methyl or when R¹ is pyrazolyl substituted with ethyl and methyl, then R² is not hydrogen, chloro, fluoro, bromo or methyl,
X₁ is NR⁵ and R¹ is methyl, methyloxy-, ethyloxy-, t-butyloxy-, benzyloxy-, phenyl or di-chloro-phenyl, then R² is not hydrogen, halo, methyl or trifluoromethyl-,
X₁ is —O— and R³ or R⁴ is a methyl, isopropyl, propyl, t-butyl or an isoxazolyl substituted with either chloro, one methyl substitutent or with one methyl and one di-chloro-phenyl substitutent, then R² is not hydrogen, chloro or methyl.
With the aforementioned provisos;
   the particular benzisoxazoles available in the Maybridge plc HTS catalog are excluded from the present set of DAAO inhibitors.

It is also an object of the present invention to provide the compounds of formula (Ic) for use as a medicine, in particular to provide the use of the compounds of formula (Ic) as DAAO inhibitors, such as for example in the manufacture of a medicament to treat mental disorders, including but not limited to a medicament to ameliorate the impaired NMDA-type glutamate receptor activity in schizophrenia patients.

In particular the compounds of formula (Ic) wherein one or more of the following restrictions apply:
those compounds of formula (Ic) wherein m is 1;
those compounds of formula (Ic) wherein X₁ represents O or NR⁵; in particular NR⁵;
those compounds of formula (Ic) wherein R¹ is NR³R⁴ or Het¹, in particular isoxazolyl or imidazolyl each independently substituted with one or more substitutents selected from C₁₋₄alkyl and phenyl substituted with one or more halo substitutents;
those compounds of formula (Ic) wherein R² is hydrogen, halo, in particular chloro or R² represents C₁₋₄alkyl, in particular methyl.
those compounds of formula (Ic) wherein R³ and R⁴ are each independently selected from hydrogen, Het² and C₁₋₄alkyl, in particular hydrogen, methyl, propyl, isopropyl or t-butyl;
those compounds of formula (Ic) wherein R³ represents hydrogen and R⁴ is C₁₋₄alkyl, phenyl or C₁₋₄alkyl substituted with phenyl;
those compounds of formula (Ic) wherein R⁵ represents hydrogen, phenyl-carbonyl- or C₁₋₄alkyl-carbonyl-;
those compounds of formula (Ic) wherein X₁ represents O and R³ and R⁴ are each independently selected from Het², Ar³, C₁₋₄alkyl or C₁₋₄alkyl substituted with one or more substitutents selected from halo, hydroxy or C₁₋₄alkyloxy-; hereinafter also referred to as the compounds of formula (Ie);
those compounds of formula (Ic) wherein Het¹ is isoxazolyl or imidazolyl each independently substituted with one or more substitutents selected from C₁₋₄alkyl and phenyl substituted with one or more halo substitutents;
those compounds of formula (Ic) wherein Het² is isoxazolyl substituted with one or more substitutents selected from C₁₋₄alkyl and phenyl substituted with one or more halo substitutents.

It is also an object of the present invention to provide the compounds of formula (Ie) for use as a medicine, in particular to provide the use of the compounds of formula (Ie) as DAAO inhibitors, such as for example in the manufacture of a medicament to treat mental disorders, including but not limited to a medicament to ameliorate the impaired NMDA-type glutamate receptor activity in schizophrenia patients.

In particular the compounds of formula (Ie) wherein one or more of the following restrictions apply:
those compounds of formula (Ie) wherein m is 1;
those compounds of formula (Ie) wherein R² is hydrogen, halo, in particular chloro or bromo or R² represents C₁₋₄alkyl, in particular methyl.
those compounds of formula (Ie) wherein R³ and R⁴ are each independently selected from hydrogen, Het² and C₁₋₄alkyl, in particular hydrogen, methyl, propyl, isopropyl or t-butyl; alternatively those compounds of formula (Ie) wherein R³ and R⁴ are each independently selected from Het² and C₁₋₄alkyl, in particular methyl, propyl, isopropyl or t-butyl;
those compounds of formula (Ie) wherein Het² is isoxazolyl substituted with one or more substitutents selected from C₁₋₄alkyl and phenyl substituted with one or more halo substitutents.

Another interesting group of compounds are those compounds of formula (I) wherein
X represents oxo and Y represents —(C═O)—R⁶, hereinafter referred to as the compounds of formula (Id) 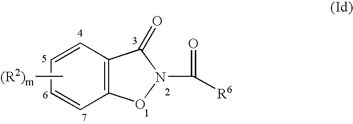
   the N-oxide forms, the pharmaceutically acceptable addition salts and the stereochemically isomeric forms thereof, wherein
m represents an integer from 1 to 3;
R⁶ represents C₁₋₄alkyl, C₁₋₄alkyloxy-, Ar⁵, Ar⁶—C₁₋₄alkyl-, —NR⁷R⁸ or Het³;
R² represents hydrogen, halo, hydroxy, nitro, cyano, hydroxycarbonyl-, amino, mono- or di (C₁₋₄alkyl)amino-, C₁₋₆alkyloxycarbonyl-, C₁₋₄alkyloxycarbonylC₁₋₄alkyloxy-, C₁₋₄alkyloxy- optionally substituted with one or more halo atoms or R² represents C₁₋₄alkyl optionally substituted with one or more halogen atoms;
R⁷ and R³ are each independently selected from hydrogen, Het⁴, Ar⁷, C₁₋₄alkyl or C₁₋₄alkyl substituted with one or more substitutents selected from halo, hydroxy or C₁₋₄alkyloxy-;
Het³ represents a heterocycle selected from oxazolyl, isoxazolyl, thiazolyl, isothiazolyl, imidazolyl, pyrazolyl, benzisoxazolyl, benzimidazolyl or benzothiazolyl wherein said heterocycle is optionally substituted with one or more substitutents each independently selected from the group consisting of amino, C₁₋₄alkyl, hydroxy-C₁₋₄alkyl-, phenyl, phenyl-C₁₋₄alkyl- and phenyl substituted with one or more halo substitutents;
Het⁴ represents a heterocycle selected from oxazolyl, isoxazolyl, thiazolyl, isothiazolyl, imidazolyl, pyrazolyl, benzisoxazolyl, benzimidazolyl or benzothiazolyl wherein said heterocycle is optionally substituted with one or more substitutents each independently selected from the group consisting of amino, C₁₋₄alkyl, hydroxy-C₁₋₄alkyl-, phenyl, phenyl-C₁₋₄alkyl- and phenyl substituted with one or more halo substitutents;
Ar⁵, Ar⁶ or Ar⁷ each independently represents phenyl optionally substituted one or where possible two or more substitutents selected from halo, nitro, C₁₋₄alkyl, hydroxy or C₁₋₄alkyloxy-.
provided that when;
   m represents 1 and R¹ represents chloro- or nitro-phenyl, then R² is not hydrogen, methoxy-, ethoxy-, chloro or fluoro;
   R¹ represents ethoxy- or methoxy-, then R² is not hydrogen, bromo, fluoro or chloro;
   R¹ represents methyl, then R² is not hydrogen, bromo or chloro.
   With the aforementioned provisos;
      the particular benzisoxazoles available in the Maybridge plc HTS catalog,
      the particular 1,2-benzisoxazoles disclosed in Science of Synthesis (2002), 11, p. 289-335,
      the particular polyamides obtained from active diacyl derivatives of 3-hydroxy-1,2-benzisoxazoles as disclosed in Journal of Polymer Science (1981), 19(5), p. 1061-1071,
      the acyl derivatives of 3-hydroxy-1,2-benzisoxazoles as disclosed in Acta Poloniae Pharmaceutica (1984), 41(6), p. 625-631; Polish Journal of Pharmacology and Pharmacy (1978), 30(5), p. 1061-1071; Polish Journal of Thermal Analysis (1979), 15(2), p. 257-260 and Chemische Berichte (1969), 102(11), p. 3775-3785, and
      the particular rearranged acyl derivatives of 3-hydroxy-1,2-benzisoxazoles as disclosed in Chemische Berichte (1970), 103(1), p. 123-132.
   are excluded from the present class of novel DAAO inhibitors.

It is also an object of the present invention to provide the compounds of formula (Id) for use as a medicine, in particular the use of the compounds of formula (Id) as DAAO inhibitors, such as for example in the manufacture of a medicament to treat mental disorders, including but not limited to a medicament to ameliorate the impaired NMDA-type glutamate receptor activity in schizophrenia patients.

In particular the compounds of formula (Id) wherein one or more of the following restrictions apply:
those compounds of formula (Id) wherein m is 1;
those compounds of formula (Id) wherein R⁶ is C₁₋₄alkyl, Ar⁵, Ar⁶—C₁₋₄alkyl- or NR⁷R⁵, in particular C₁₋₄alkyl, phenyl or phenyl-C₁₋₄alkyl-;
those compounds of formula (Id) wherein R² is hydrogen, halo or C₁₋₄alkyl, in particular methyl or chloro.
those compounds of formula (Id) wherein R⁷ and R⁸ are each independently selected from hydrogen, Het⁴ and C₁₋₄alkyl, in particular hydrogen, methyl, propyl, Het⁴, isopropyl or t-butyl;
those compounds of formula (Id) wherein Het⁴ is isoxazolyl or imidazolyl each independently substituted with one or more substitutents selected from C₁₋₄alkyl and phenyl substituted with one or more halo substitutents;
   in particular the aforementioned group of compounds for use as a medicine, even more particular for the use in the manufacture of a medicament for treating mental disorders as described hereinbefore, such as for example in the manufacture of a medicament for treating schizophrenia.

A preferred group of compounds consists of those compounds of formula (I) wherein one or more of the following restrictions apply:
those compounds of formula (I) wherein X represents -oxo;
those compounds of formula (I) wherein R² represents hydrogen or halo, in particular hydrogen or chloro;
those compounds of formula (I) wherein R⁶ represents Ar⁵, Ar⁶—C₁₋₄alkyl-, —NR⁷R⁸ or Het³, in particular phenyl, benzyl, isoxazolyl substituted with methyl and dichlorophenyl or R⁶ represents NR⁷R⁸;
those compounds of formula (I) wherein R⁷ and R⁸ are each independently selected from hydrogen, Het⁴ or C₁₋₄alkyl;
those compounds of formula (I) wherein Het⁴ represents isoxazolyl or imidazolyl each independently substituted with one or more substitutents selected from C₁₋₄alkyl and phenyl substituted with one or more halo substitutent.

Other special group of compounds are those compounds wherein one or more of the following restrictions apply;
those compounds of formula (I) wherein X is -Z-R¹;
those compounds of formula (I) wherein Z is oxycarbonyl;
those compounds of formula (I) wherein R¹ is mono- or di(methyl)amino-;
those compounds of formula (I) wherein R¹ is Het¹, in particular isoxazolyl substituted with methyl;
those compounds of formula (I) wherein R² is trifluoromethyl or halo, in particular chloro;
those compounds of formula (I) wherein R³ and/or R⁴C₁₋₄alkyl, in particular methyl, propyl, isopropyl or t-butyl;
those compounds of formula (I) wherein Z represents -oxy-carbonyl or —NR⁵-carbonyl and R¹ is —NR³R⁴ or Het¹;
   in particular the aforementioned group of compounds for use as a medicine, even more particular for the use in the manufacture of a medicament for treating mental disorders such as for example schizophrenia.

In a further embodiment of the present invention the R² substitutent is at position 5 or 6, the Het¹ substitutent is 2′-isoxazolyl optionally substituted with methyl, in particular substituted at position 5′ of said isoxazolyl substitutent.

It is also an object of the present invention to provide compounds of formula (I) wherein R¹ is a heterocycle Het¹ selected from the group consisting of isoxazolyl, pyrazolyl or benzisoxazolyl wherein said Het¹ is optionally substituted with one or more substitutents each independently selected from the group consisting of C₁₋₄alkyl, phenyl and phenyl substituted with one or more halo substitutents, provided that when R¹ is a substituted isoxazolyl, R² is not chloro.

As further exemplified in the experimental part of the description, the compounds of formula (Ia), (Ib), (Ie) or (If) wherein R², R³, R⁴, R⁷ and R⁸ are defined as hereinbefore, are generally prepared using the following synthesis scheme.

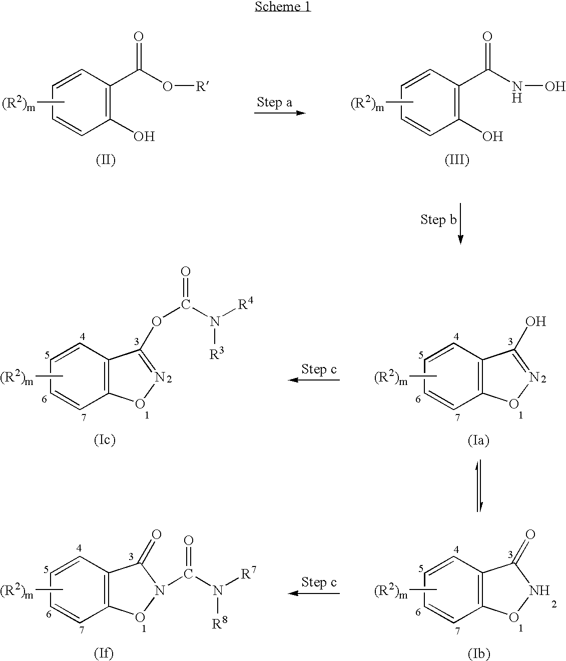

R², R³, R⁴, R⁷, and R⁸ are defined as for the compounds of formula (I) hereinbefore

The compounds of formula (II) wherein —(C═O)—O—R′ represents an ester residue and which are starting materials of this invention are either a well known compounds or can be synthesized according to a well known method [e.g. Chem. Abstr., 49, 11594(1955), J. Org. Chem., 44, 3292(1979) or Chem. Ber., 100, 954(1967)]. In a first step (Step a), the compound of formula (II) is treated with hydroxylamine in an inert solvent, such as for example dichloromethane, in the presence of a base to prepare a compound having the general formula (III).

The solvent used is not particularly restricted provided that it does not interfere with the reaction and can dissolve a certain amount of the starting material and it may be, for example, hexane, toluene, diisopropyl ether or tetrahydrofuran.

The base used may for example be, an alkali metal carbonate such as sodium carbonate, potassium carbonate or lithium carbonate, an alkali metal hydrogen carbonate such as sodium hydrogen carbonate, potassium hydrogen carbonate or lithium hydrogen carbonate, an alkali metal hydride such as potassium hydride, sodium hydride or lithium hydride or an alkali metal hydroxide such as sodium hydroxide, potassium hydroxide or lithium hydroxide.

The reaction temperature may be altered depending on the starting material or reagents but is usually in the range from 0° C. to 100° C. and preferably from 20° C. to 50° C.

The reaction time may be altered depending on the starting material, reagents or reaction temperature but it is usually between 10 minutes and 10 hours and preferably between 30 minutes and 5 hours.

After completion of the reaction, the target compound of this process is isolated from the reaction mixture according to a usual method. For example, the solvent is removed by evaporation, adding an acidic aqueous solution to the residue (e.g. using hydrochloric acid), filter the precipitated compound, washing the extract with water and drying it under reduced pressure at an elevated temperature (e.g. in the range of 50° C. to 150° C.). The target compound obtained may be, if necessary, purified by recrystallization, reprecipitation or chromatography.

In a second step (Step b), the compound having the general formula (Ia) is synthesized from that having the general formula (II) according to the art known cyclization reaction wherein the compound of formula (II) is treated in an inert solvent, such as for example tetrahydrofuran, dioxane or diisopropyl ether, with a dehydrating agent, such as for example, dicyclohexylcarbodiimide (DCC), chlorosilanes and N,N′-carbonyldiimidazole (CDI), to prepare the benzisoxazoles of general formula (Ia) and its tautomeric form (Ib).

The reaction temperature may be altered depending on the starting material or reagents but is usually in the range from 0° C. to 100° C. and preferably from 20° C. to 70° C.

The reaction time may be altered depending on the starting material, reagents or reaction temperature but it is usually between 10 minutes and 10 hours and preferably between 30 minutes and 5 hours.

After completion of the reaction, the target compound of this process is isolated from the reaction mixture according to a usual method. For example, the solvent is removed by evaporation, adding an acidic aqueous solution to the residue (e.g. using hydrochloric acid), filter the precipitated compound, washing the extract with water and drying it under reduced pressure at an elevated temperature (e.g. in the range of 50° C. to 150° C.). The target compound obtained may be, if necessary, purified by recrystallization, reprecipitation or chromatography.

In a final step (Step c), the carbamate esters of formula (Ie) and the ureas of formula (If) are synthesized from the benzisoxazole (Ia) and its respective tautomeric form (Ib) according to the art known reaction with isocyanates [see for example, Advanced Organic Chemistry Reactions, Mechanisms and Structures, March J., Ed., 791 (1985) John Wiley & Sons, Inc., New York USA; Introduction to Organic Chemistry, Streitweiser A. and Heathcock C. H., Ed., 785 (1981) MacMillan Publishing Co., Inc., New York USA]. Optionally, the addition of alcohols to isocyanates can also be catalyzed by organometallic compounds [J. Chem. Soc., C 2663, 1479(1968)], by light [J. Org. Chem., 42, 1428(1977)], or, for tertiary alcohols by lithium alkoxides [J. Org. Chem., 43, 2690(1978)]. This reaction is usually performed in an inert solvent such as for example, triethylamine, dioxine, diisopropylether, tetrahydrofuran or methylenechloride. The reaction temperature and reaction time may be altered depending on the starting material or reagents but is usually performed overnight at room temperature.

After completion of the reaction, the target compound of this process is isolated from the reaction mixture according to a usual method. For example, the target compound is isolated by filtering the target product precipitated in the reaction mixture, or neutralizing the reaction mixture followed by addition of a hydrophobic solvent (e.g. benzene, ether, ethyl acetate) to extract the compound, washing the organic layer with water, drying it either at reduced pressure and elevated temperature or over anhydrous magnesium sulphate and removing the solvent by evaporation. The target compound obtained may be, if necessary, purified by recrystallization, reprecipitation or chromatography.

As further exemplified in the experimental part of the description, the compounds of formula (Ig), (Ih) and (Ii) wherein R², R³, R⁴ are defined as hereinbefore, are generally prepared from the corresponding 3-aminobenzisoxazole (Ig) using art known reaction conditions (Scheme 2). 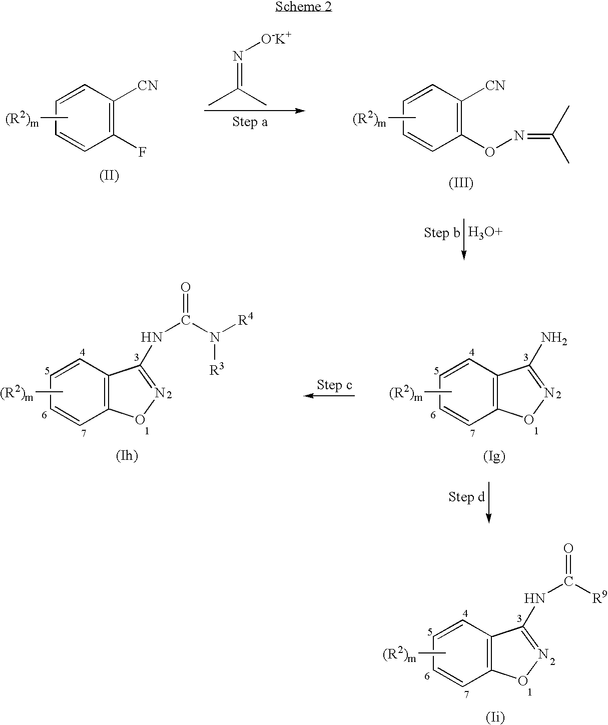
R², R³ and R⁴ are defined as for the compounds of formula (I) hereinbefore;
R⁹ represents C₁₋₄alkyl, Ar¹, Ar²—C₁₋₄alkyl or Het¹, wherein Ar¹, Ar² and Het¹ are defined as for the compounds of formula (I).

The 3-aminobenzisoxazole is generally prepared using for example the two-step Shutske's synthesis as described in J. Heterocycl. Chem. (1989) 26, 1293. The Shutske's synthesis of 3-aminobenzisoxazoles, involves acetone oxime addition to the appropriate 2-fluorobenzonitrile (Step a), followed by a subsequent acid-mediated cyclization, using for example hydrochloric acid (Step b).

The urea derivatives of formula (Ih) are generally obtained according to the art known reaction with isocyanates (Step c) [see for example, Advanced Organic Chemistry: Reactions, Mechanisms and Structures, March J., Ed., 791 (1985) John Wiley & Sons, Inc., New York USA; Introduction to Organic Chemistry, Streitweiser A. and Heathcock C. H., Ed., 785 (1981) MacMillan Publishing Co., Inc., New York USA]. This reaction is usually performed in an inert solvent such as for example, diisopropylether, tetrahydrofuran or methylenechloride. The reaction temperature and reaction time may be altered depending on the starting material or reagents but is usually performed overnight at room temperature.

The amides of formula (II) are generally obtained according to the art known reaction with the corresponding carboxylic acids or acylhalides (Step d) [see for example, Introduction to Organic Chemistry, Streitweiser A. and Heathcock C. H., Ed., 548 (1981) MacMillan Publishing Co., Inc., New York USA].

Where necessary or desired, any one or more of the following further steps in any order may be performed:
(i) removing any remaining protecting group(s);
(ii) converting a compound of formula (I) or a protected form thereof into a further compound of formula (I) or a protected form thereof;
(iii) converting a compound of formula (I) or a protected form thereof into a N-oxide, a salt, a quaternary amine or a solvate of a compound of formula (I) or a protected form thereof;
(iv) converting a N-oxide, a salt, a quaternary amine or a solvate of a compound of formula (I) or a protected form thereof into a compound of formula (I) or a protected form thereof;
(v) converting a N-oxide, a salt, a quaternary amine or a solvate of a compound of formula (I) or a protected form thereof into another N-oxide, a pharmaceutically acceptable addition salt a quaternary amine or a solvate of a compound of formula (I) or a protected form thereof;
(vi) where the compound of formula (I) is obtained as a mixture of (R) and (S) enantiomers resolving the mixture to obtain the desired enantiomer.

Compounds of formula (I), N-oxides, addition salts, quaternary amines and stereochemical isomeric forms thereof can be converted into further compounds according to the invention using procedures known in the art.

It will be appreciated by those skilled in the art that in the processes described above the functional groups of intermediate compounds may need to be blocked by protecting groups.

Functional groups which it is desirable to protect include hydroxy, amino and carboxylic acid. Suitable protecting groups for hydroxy include trialkylsilyl groups (e.g. tert-butyldimethylsilyl, tert-butyldiphenylsilyl or trimethylsilyl), benzyl and tetrahydropyranyl. Suitable protecting groups for amino include tert-butyloxycarbonyl or benzyloxycarbonyl. Suitable protecting groups for carboxylic acid include C₍₁₋₆₎alkyl or benzyl esters.

The protection and deprotection of functional groups may take place before or after a reaction step.

The use of protecting groups is fully described in ‘Protective Groups in Organic Synthesis’ 3^{rd} edition, T W Greene & P G M Wutz, Wiley Interscience (1998)

Additionally, the N-atoms in compounds of formula (I) can be methylated by art-known methods using CH₃—I in a suitable solvent such as, for example 2-propanone, tetrahydrofuran or dimethylformamide.

The compounds of formula (I) can also be converted into each other following art-known procedures of functional group transformation of which some examples are mentioned hereinafter.

The compounds of formula (I) may also be converted to the corresponding N-oxide forms following art-known procedures for converting a trivalent nitrogen into its N-oxide form. Said N-oxidation reaction may generally be carried out by reacting the starting material of formula (I) with 3-phenyl-2-(phenylsulfonyl)oxaziridine or with an appropriate organic or inorganic peroxide. Appropriate inorganic peroxides comprise, for example, hydrogen peroxide, alkali metal or earth alkaline metal peroxides, e.g. sodium peroxide, potassium peroxide; appropriate organic peroxides may comprise peroxy acids such as, for example, benzenecarboperoxoic acid or halo substituted benzenecarboperoxoic acid, e.g. 3-chlorobenzenecarboperoxoic acid, peroxoalkanoic acids, e.g. peroxoacetic acid, alkylhydroperoxides, e.g. t-butyl hydroperoxide. Suitable solvents are, for example, water, lower alkanols, e.g. ethanol and the like, hydrocarbons, e.g. toluene, ketones, e.g. 2-butanone, halogenated hydrocarbons, e.g. dichloromethane, and mixtures of such solvents.

Pure stereochemically isomeric forms of the compounds of formula (I) may be obtained by the application of art-known procedures. Diastereomers may be separated by physical methods such as selective crystallization and chromatographic techniques, e.g. counter-current distribution, liquid chromatography and the like.

Some of the compounds of formula (I) and some of the intermediates in the present invention may contain an asymmetric carbon atom. Pure stereochemically isomeric forms of said compounds and said intermediates can be obtained by the application of art-known procedures. For example, diastereoisomers can be separated by physical methods such as selective crystallization or chromatographic techniques, e.g. counter current distribution, liquid chromatography and the like methods. Enantiomers can be obtained from racemic mixtures by first converting said racemic mixtures with suitable resolving agents such as, for example, chiral acids, to mixtures of diastereomeric salts or compounds; then physically separating said mixtures of diastereomeric salts or compounds by, for example, selective crystallization or chromatographic techniques, e.g. liquid chromatography and the like methods; and finally converting said separated diastereomeric salts or compounds into the corresponding enantiomers. Pure stereochemically isomeric forms may also be obtained from the pure stereochemically isomeric forms of the appropriate intermediates and starting materials, provided that the intervening reactions occur stereospecifically.

An alternative manner of separating the enantiomeric forms of the compounds of formula (I) and intermediates involves liquid chromatography, in particular liquid chromatography using a chiral stationary phase.

Some of the intermediates and starting materials as used in the reaction procedures mentioned hereinabove are known compounds and may be commercially available or may be prepared according to art-known procedures.

As described in the experimental part hereinafter, the DAAO inhibitory activity of the compounds of formula (I) and the intermediates of formula (Ia) and (Ig) has been demonstrated in vitro, in enzymatic assays to measure the catalytic activity of DAAO.

Accordingly, the present invention provides the compounds of formula (I), the intermediates of formula (Ia), the intermediates of formula (Ig) and their pharmaceutically acceptable N-oxides, addition salts, quaternary amines and stereochemically isomeric forms for use as a medicine or in therapy. More particular in the treatment of mental disorders including schizophrenia. The compounds of formula (I), the intermediates of formula (Ia), the intermediates of formula (Ig) and their pharmaceutically acceptable N-oxides, addition salts, quaternary amines and the stereochemically isomeric forms may hereinafter be referred to as compounds according to the invention.

Disorders for which the compounds according to the invention are particularly useful are schizophrenia and other diseases linked to NMDA receptor dysfunction including pain, spasticity, epilepsy, and diseases with impaired learning and memory such as Alzheimer's disease, Huntington's disease, Parkinson's disease, AIDS, attention deficit disorder, attention deficit hyperactivity disorder, and autism. Likewise, diseases caused by brain damage such as trauma or stroke may benefit. As mentioned hereinbefore, the compounds of the present invention are effective in combating the negative symptoms in schizophrenia, i.e. the impaired social interaction (e.g. impoverishment of affect, thought, and initiative) and the cognitive disturbances of schizophrenic patients. Dopamine antagonists are reportedly effective in combating the positive symptoms in schizophrenia, i.e. psychoses, aggressive behaviour and anxiety. Hence, the compounds of the present invention are especially interesting for use in a combination therapy combining a DAAO inhibitor with a dopamine inhibitor to offer relief of both the positive and negative symptoms of schizophrenia.

In view of the utility of the compounds according to the invention, there is provided a method for the treatment of an animal, for example, a mammal including humans, suffering from a mental disorder such as schizophrenia and the other disease conditions mentioned above, which comprises administering an effective amount of a compound according to the present invention.

Said method comprising the systemic or topical administration of an effective amount of a compound according to the invention, to warm-blooded animals, including humans. One skilled in the art will recognize that a therapeutically effective amount of the DAAO inhibitors according to the invention, is an amount sufficient to reduce DAAO activity and hence to ameliorate the impaired NMDA-type glutamate receptor activity in schizophrenia patients. This amount varies inter alia, depending on the level of impaired NMDA-type glutamate receptor activity, the concentration of the compound in the therapeutic formulation and the condition of the patient. Generally, an amount of DAAO inhibitor to be administered as a therapeutic agent for treating mental disorders, such as for example schizophrenia, will be determined on a case-by-case basis by an attending physician.

Generally, a suitable dose is one that results in a concentration of the DAAO inhibitor at the treatment site in the range of 0.5 nM to 200 μM, and more usual 5 nM to 50 μM. To obtain these treatment concentrations, a patient in need of treatment likely will be administered between 0.01 mg/kg to 300 mg/kg body weight, in particular from 10 mg/kg to 100 mg/kg body weight. As noted above, the amounts may vary on a case-by-case basis. In these methods of treatment the compounds according to the invention are preferably formulated prior to admission. As described herein below, suitable pharmaceutical formulations are prepared by known procedures using well known and readily available ingredients.

In yet a further aspect, the present invention provides the use of the compounds according to the invention in the manufacture of a medicament for treating any of the aforementioned mental disorders or indications.

The amount of a compound according to the present invention, also referred to here as the active ingredient, which is required to achieve a therapeutical effect will be, of course, vary with the particular compound, the route of administration, the age and condition of the recipient, and the particular disorder or disease being treated. A suitable daily dose would be from 0.01 mg/kg to 50 mg/kg body weight, in particular from 0.05 mg/kg to 10 mg/kg body. A method of treatment may also include administering the active ingredient on a regimen of between one and four intakes per day.

While it is possible for the active ingredient to be administered alone, it is preferable to present it as a pharmaceutical composition. Accordingly, the present invention further provides a pharmaceutical composition comprising a compound according to the present invention, together with a pharmaceutically acceptable carrier or diluent. The carrier or diluent must be “acceptable” in the sense of being compatible with the other ingredients of the composition and not deleterious to the recipients thereof.

The pharmaceutical compositions of this invention may be prepared by any methods well known in the art of pharmacy, for example, using methods such as those described in Gennaro et al. Remington's Pharmaceutical Sciences (18^{th} ed., Mack Publishing Company, 1990, see especially Part 8: Pharmaceutical preparations and their Manufacture). A therapeutically effective amount of the particular compound, in base form or addition salt form, as the active ingredient is combined in intimate admixture with a pharmaceutically acceptable carrier, which may take a wide variety of forms depending on the form of preparation desired for administration. These pharmaceutical compositions are desirably in unitary dosage form suitable, preferably, for systemic administration such as oral, percutaneous, or parenteral administration; or topical administration such as via inhalation, a nose spray, eye drops or via a cream, gel, shampoo or the like. For example, in preparing the compositions in oral dosage form, any of the usual pharmaceutical media may be employed, such as, for example, water, glycols, oils, alcohols and the like in the case of oral liquid preparations such as suspensions, syrups, elixirs and solutions: or solid carriers such as starches, sugars, kaolin, lubricants, binders, disintegrating agents and the like in the case of powders, pills, capsules and tablets. Because of their ease in administration, tablets and capsules represent the most advantageous oral dosage unit form, in which case solid pharmaceutical carriers are obviously employed. For parenteral compositions, the carrier will usually comprise sterile water, at least in large part, though other ingredients, for example, to aid solubility, may be included. Injectable solutions, for example, may be prepared in which the carrier comprises saline solution, glucose solution or a mixture of saline and glucose solution. Injectable suspensions may also be prepared in which case appropriate liquid carriers, suspending agents and the like may be employed. In the compositions suitable for percutaneous administration, the carrier optionally comprises a penetration enhancing agent and/or a suitable wettable agent, optionally combined with suitable additives of any nature in minor proportions, which additives do not cause any significant deleterious effects on the skin. Said additives may facilitate the administration to the skin and/or may be helpful for preparing the desired compositions. These compositions may be administered in various ways, e.g., as a transdermal patch, as a spot-on or as an ointment. As appropriate compositions for topical application there may be cited all compositions usually employed for topically administering drugs e.g. creams, jellies, dressings, shampoos, tinctures, pastes, ointments, salves, powders and the like. Application of said compositions may be by aerosol, e.g. with a propellant such as nitrogen, carbon dioxide, a freon, or without a propellant such as a pump spray, drops, lotions, or a semisolid such as a thickened composition which can be applied by a swab. In particular, semisolid compositions such as salves, creams, jellies, ointments and the like will conveniently be used.

It is especially advantageous to formulate the aforementioned pharmaceutical compositions in dosage unit form for ease of administration and uniformity of dosage. Dosage unit form as used in the specification and claims herein refers to physically discrete units suitable as unitary dosages, each unit containing a predetermined quantity of active ingredient calculated to produce the desired therapeutic effect in association with the required pharmaceutical carrier. Examples of such dosage unit forms are tablets (including scored or coated tablets), capsules, pills, powder packets, wafers, injectable solutions or suspensions, teaspoonfuls, tablespoonfuls and the like, and segregated multiples thereof.

In order to enhance the solubility and/or the stability of the compounds of formula (I) in pharmaceutical compositions, it can be advantageous to employ α-, β- or γ-cyclodextrins or their derivatives. Also co-solvents such as alcohols may improve the solubility and/or the stability of the compounds of formula (I) in pharmaceutical compositions. In the preparation of aqueous compositions, addition salts of the subject compounds are obviously more suitable due to their increased water solubility.

### EXPERIMENTAL PART

Hereinafter, the term ‘RT’ means room temperature, ‘THF’ means tetrahydrofuran, DIPE means diisopropyl ether, DMF means dimethylformamide, CDI means N,N′-carbonyldiimidazole, KtBuO means 2-Propanol, 2-methyl-, potassium salt.

### A. Preparation of the Intermediates

### Example A1

a) Preparation of

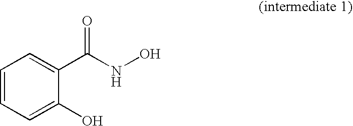

A solution of hydroxylamine (3M) was stirred at RT under N₂-atmosphere, then NaOH (7.05 mol in 240 ml H₂O) and methyl salicylate (300 g in 750 ml dioxane) were added dropwise and the reaction mixture was stirred for 12 hours at RT. After completion, the reaction solvent was evaporated at 50° C., the remaining residue cooled and acidified with 12N HCl. The mixture was stirred for 30 min. at 10-15° C. and the resulting precipitate filtrated, washed with ice-water and dried under reduced pressure at 90° C. Quantitative Yielding 2-Hydroxybenzhydroxamic Acid (intermediate 1).
b) Preparation of

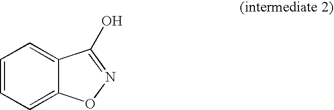

A solution of intermediate 1 (1.92 M in THF) was stirred at 60° C. A solution of CDI (3.84 M in THF) was added over 30 min. under reflux to the aforementioned solution and refluxed for another 2 hours at 60° C. The reaction mixture was cooled to 40° C. and the solvent evaporated. After completion, the remaining residue was quenched with water and acidified with 12N HCl to pH 2. The mixture was stirred for 30 min. at 10-15° C. and the resulting precipitate filtrated, washed with ice-water and dried under reduced pressure at 90° C. Quantitative Yielding 3-Hydroxybenzisoxazole (intermediate 2).

### Example A2

Preparation of

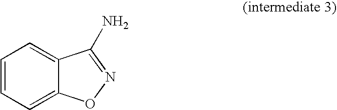

A solution of acetoxime (0.055 mol) in DMF (50 ml) was stirred at RT, then KtBuO (0.055 mol) was added and the solution stirred for another 30 min. at RT. 2-cyanofluorobenzene (0.050 mol) was added drop wise and the reaction mixture stirred for another hour at RT. The reaction mixture was poured out in a solution of 200 ml isopropylether and 200 ml saturated NH₄Cl solution and stirred vigorously for 10 minutes. After completion, the organic layer was separated, washed with water, dried (MgSO₄), filtered off and the solvent was evaporated dry. The residue (Yield: 9 g) was dissolved in ethanol (100 ml). A solution of 2N HCl (100 ml) was added and the reaction mixture refluxed for 1 hour. After completion the solvent was evaporated, the aqueous residue alkanized with a solution of K₂CO₃ and extracted with EtOAc. The organic layer was separated, washed with water, dried (MgSO₄), filtered off and the solvent was evaporated dry. Quantitative Yielding 1-2-Benzisoxazole-3-amine 4.7 g (70%), Melting Point 111° C. (intermediate 3).

### B. Preparation of the Compounds

### Example B1

Preparation of

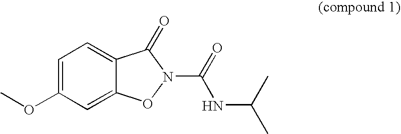

A solution of 3-hydroxy-6-methoxy-1,2-benzisoxazole (0.0007 mol) in dioxane (5 ml) was stirred at RT and then isopropyl isocyanate (0.0010 mol) was added dropwise. The reaction mixture was stirred overnight at RT. The reaction was completed and the mixture was evaporated dry. The residue was recrystallized in DIPE. Yielding 0.083 g compound 1 (yield of 47%), melting point 121° C.

### Example B2

Preparation of 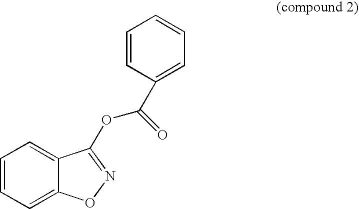
Preparation of

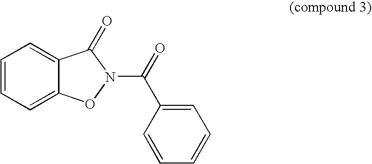

Triethylamine (0.0025 mol) was added to a solution of intermediate 2 in CH₂Cl₂ (5 ml). The reaction mixture was stirred at RT and then benzoylchloride (0.0025 mol) was added dropwise. The reaction mixture was stirred overnight at RT. The mixture washed 2 times with H₂O. The organic layer was separated, dried (MgSO₄), filtered off and the solvent was evaporated dry. The residue was purified by column chromatography over silicagel (eluent CH₂Cl₂) yielding 0.430 g compound 2 (yield of 72%, Melting Point 65° C.) and 0.030 g compound 3 (yield of 5%, Melting Point 115° C.)

### Example B3

Preparation of

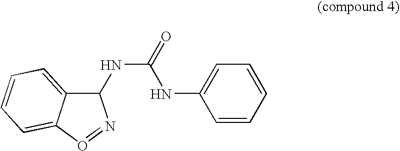

To a solution of intermediate 3 (0.0025 mol) in isopropylether (5 ml) THF (1 ml) was added and the reaction mixture stirred at RT. Phenylisocyanaat (0.0050 mol) was added and the reaction mixture stirred overnight at RT. The precipitate was filtered off, washed with isopropylether and evaporated dried. The residue was further purified over reversed phase HPLC on a Xterra MS C18 column (3.5 μm, 4.6×100 mm) with a flow rate of 1.6 ml/min (Elution conditions: three mobile phases (mobile phase A 95% 25 mM ammoniumacetate+5% acetonitrile; mobile phase B: acetonitrile; mobile phase C: methanol) were employed to run a gradient condition from 100% A to 50% B and 50% C in 6.5 min., to 100% B in 1 min, 100% B for 1 min. and re-equilibrate with 100% A for 1.5 min.) yielding 0.010 g of compound 4 (yield of 5%, Melting Point 246° C.).

### Example B4

Preparation of

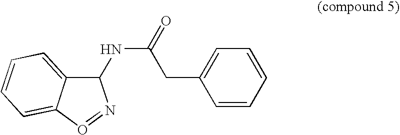

A mixture of intermediate 3 (0.0025 mol) and triethylamine (0.0025 mol) in CH₂Cl₂ (10 ml) was stirred at RT. Phenylacetylchloride (0.0025 mol) was added drop wise and the reaction mixture stirred overnight at RT. The reaction mixture washed 2 times with H₂O. The organic layer was separated, dried (MgSO₄), filtered off and the solvent was evaporated. The residue was crystallized in isopropanol. Yielding 0.210 g compound 5 (yield of 84%), melting point 164° C.

### Example B5

Preparation of

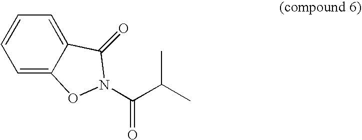

A mixture of intermediate 2 (0.0025 mol) and triethylamine (0.0025 mol) in CH₂Cl₂ (5 ml) was stirred at RT. 2-methylpropanoyl chloride (0.0025 mol) was added drop wise and the reaction mixture stirred overnight at RT. The reaction mixture washed 2 times with H₂O. The organic layer was separated, dried (MgSO₄), filtered off and the solvent was evaporated dry. The residue was further purified using column chromatography over silicagel (eluent: hexane/CH₂Cl₂ 60/40) yielding 0.300 g of compound 6 (yield of 59%, Melting Point 88° C.).

### Example B6

Preparation of 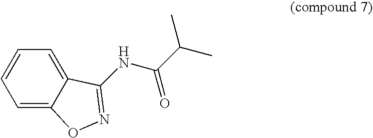
Preparation of

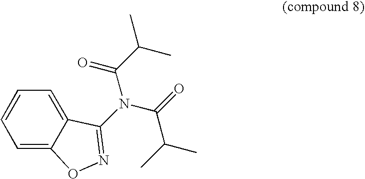

A mixture of intermediate 3 (0.0025 mol) and triethylamine (0.0025 mol) in CH₂Cl₂ (10 ml) was stirred at RT. 2-methylpropanoyl chloride (0.0025 mol) was added dropwise and the reaction mixture stirred overnight at RT. The reaction mixture was washed 2 times with H₂O. The organic layer was separated, dried (MgSO₄), filtered off and the solvent was evaporated dry. The residue was further purified by column chromatography over silicagel (eluent: CH₂Cl₂) yielding 0.235 g of compound 8 and a fraction which was further purified using reversed phase HPLC chromatography on a Xterra MS C18 column (3.5 μm, 4.6×100 mm) with a flow rate of 1.6 ml/min (Elution conditions: three mobile phases (mobile phase A 95% 25 mM ammoniumacetate+5% acetonitrile; mobile phase B: acetonitrile; mobile phase C: methanol) were employed to run a gradient condition from 100% A to 50% B and 50% C in 6.5 min., to 100% B in 1 min, 100% B for 1 min. and re-equilibrate with 100% A for 1.5 min.) yielding 0.010 g of compound 7 (Melting Point 130° C.).

### Example B7

Preparation of 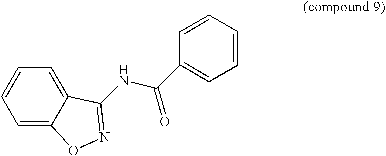
Preparation of 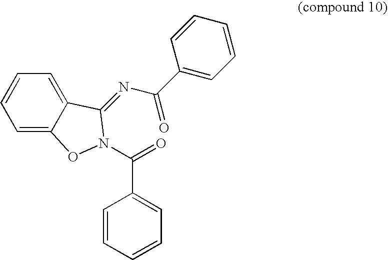
Preparation of

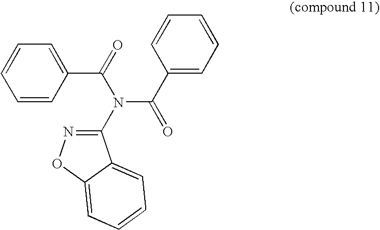

To a solution of triethylamine (0.0025 mol) in CH₂Cl₂ (5 ml) benzoylchloride (0.0025 mol) was added. The mixture stirred at RT and intermediate 3 (0.0025 mol) in CH₂Cl₂ (5 ml) added dropwise. The reaction mixture was stirred overnight at RT. The mixture washed 2 times with H₂O. The organic layer was separated, dried (MgSO₄), filtered off and the solvent was evaporated. The residue was purified by column chromatography over silicagel (eluent CH₂Cl₂) yielding 0.010 g compound 9 (yield of 2%, Melting Point 133° C.) and a fraction which was further purified by column chromatography over silicagel (eluent hexane/CH₂Cl₂) yielding 0.225 g of compound 10 (yield of 38%, Melting Point 97° C.-100° C.), and 0.100 g of compound 11 (yield of 17%, Melting Point 154° C.-160° C.).

The table herein below, provides further compounds made according to example A1. These compounds are intermediate compounds for the synthesis of the corresponding carbamates and esters according to examples B1 and B2 respectively, but were shown to have DAAO inhibiting activity and are accordingly useful as active compounds in the manufacture of a medicament, in particular a medicament for the treatment of schizophrenia and the other disease conditions mentioned hereinbefore.

TABLE 1

| | |
|---|---|
| | (Ia) |
| 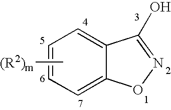 | |

| Int. No. | m | R² | melting point |
|---|---|---|---|
| 4 | 1 | 5-Cl | 216° C. |
| 5 | 1 | 6-Cl | — |
| 6 | 1 | 5-NO₂ | 201° C. |
| 7 | 1 | 5-methoxy | 181° C. |
| 8 | 1 | 6-methoxy | 208° C. |
| 9 | 1 | 4-F | 76-86° C. |
| 10 | 1 | 5-methyl | 154° C. |
| 11 | 1 | 6-methyl | — |
| 12 | 1 | 5-F | 68-78° C. |
| 13 | 1 | 5-I | 108-118° C. |
| 14 | 1 | 5-Br | 90-100° C. |
| 15 | 1 | 6-trifluoromethyl | 62-72° C. |

The tables 2 and 3 herein below, provides further compounds made according to examples B2 given herein before. These compounds were shown to have DAAO inhibiting activity and are accordingly useful as active compounds in the manufacture of a medicament, in particular a medicament for the treatment of schizophrenia and the other disease conditions mentioned hereinbefore.

TABLE 2

| | |
|---|---|
| | (Ie) |
| 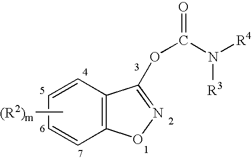 | |

| Co. No. | m | R² | R³ | R⁴ | melting point |
|---|---|---|---|---|---|
| 12 | 0 | — | -methyl | -methyl | |
| 13 | 1 | 5-methyl | -methyl | -methyl | |
| 14 | 1 | 6-Cl | -methyl | -methyl | |
| 15 | 1 | 5-Cl | -methyl | -methyl | — |

TABLE 3

| | |
|---|---|
| | (Ic) |
| 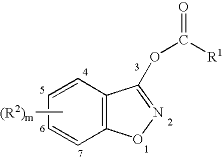 | |

| Co. No. | m | R² | R¹ | melting point |
|---|---|---|---|---|
| 16 | 1 | 6-Cl | 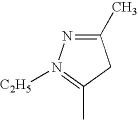 | — |
| 17 | 1 | 5-Cl | 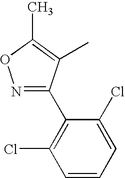 | — |
| 18 | 1 | 6-Cl | 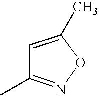 | |
| 19 | 1 | 5-methyl | 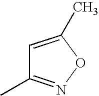 | 126-128° C. |
| 20 | 1 | — | 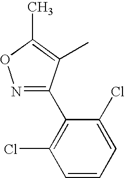 | — |

Table 4 herein below, provides further compounds made according to example B1 given herein before. These compounds were shown to have DAAO inhibiting activity and are accordingly useful as active compounds in the manufacture of a medicament, in particular a medicament for the treatment of schizophrenia and the other disease conditions mentioned herein before.

TABLE 4

| | |
|---|---|
| | (If) |
| 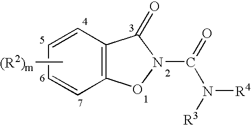 | |

| Co. | Ex. | | | | | |
|---|---|---|---|---|---|---|
| No. | No. | m | R² | R³ | melting point | |
| 21 | B1 | 1 | 5-Cl | —H | 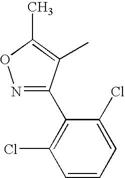 | — |
| 22 | B1 | 1 | 5-Cl | —H | -methyl | 178-180° C. |
| 23 | B1 | 1 | 5-Cl | —H | -isopropyl | 149.4- |
| | | | | | | 154.1° C. |
| 24 | B1 | 1 | 6-Cl | —H | -isopropyl | — |
| 25 | B1 | 1 | 5-Cl | —H | -propyl | 134.0- |
| | | | | | | 136.2° C. |
| 26 | B1 | 1 | 5-methyl | —H | -t-butyl | — |
| 27 | B1 | 1 | 6-Cl | —H | -t-butyl | — |
| 28 | B1 | 1 | 5-methyl | —H | -isopropyl | 105-107° C. |
| 29 | B1 | 0 | — | —H | -isopropyl | — |

Table 5 herein below, provides further compounds made according to example B2 given herein before. These compounds were shown to have DAAO inhibiting activity and are accordingly useful as active compounds in the manufacture of a medicament, in particular a medicament for the treatment of schizophrenia and the other disease conditions mentioned herein before.

TABLE 5

| | |
|---|---|
| | (Id) |
| 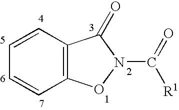 | |

| Co. No. | Ex. No. | R¹ | melting point |
|---|---|---|---|
| 3 | B2 | -phenyl | 115° C. |
| 30 | B2 | 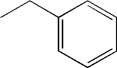 | 91° C. |

### C. Pharmacological Examples

D-amino acid oxidase (DAAO; EC 1.4.3.3) catalyzes the oxidation of D-stereo isomers of amino acids.

The general reaction can be described as:

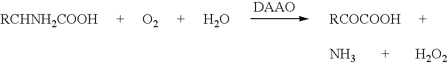

Two in vitro methods were developed to measure the catalytic activity of DAAO:
1. The peroxide method: measures the amount of peroxide produced, using an auxiliary enzyme horse radish peroxidase
2. The keto acid method: measures the ax keto acid formed

Both methods are adapted from:

Nagata, Y, Shimojo, J., Akino, K. Two spectrophotometric assays for D-amino acid oxidase: for study of distribution patterns. Int. J. Biochem 20, (1988) p 1235-1238

### Example C.1

### In Vitro Inhibition of DAAO Using the Peroxide Method

The general reaction to determine the amount of peroxide produced by catalytic action of DAAO can be described as:

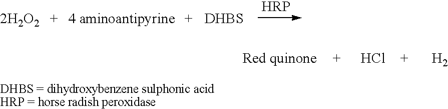

DAAO (2.6 μg/ml) is incubated for 60 minutes at room temperature with the substrate D-Alanine (7.5 mM) in 0.019 M sodiumpyrophosphate buffer containing FAD(5.5 μg/ml), HRP (200 μg/ml), DHBS (1667 μg/ml) and 4-aminoantipyrine(500 μg/ml) in a total volume of 50 μl. Compounds were added in a 0.5 μl volume to a final DMSO concentration of 1%. The reaction was terminated by addition of 30 μl of 0.5M phosphate buffer pH 5.0. The quinone-imine dye was detected by measuring the absorbance at 492 nm. All products were purchased from Sigma.

### Example C.2

### In Vitro Inhibition of DAAO Using the Keto Acid Method

The general reaction to determine the amount of peroxide produced by catalytic action of DAAO can be described as:

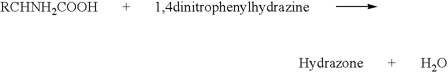

DAAO (20 μg/ml) is incubated for 10 min at room temperature with the substrate D-Alanine (15 mM) in 0.19 mM pyrophosphatebuffer pH 8.3 containing FAD (11 μg/ml), and bovine liver catalase (EC 1.11.1.6) (4.3 mg/ml) in a total volume of 50 μl. Compounds were added in a 0.5 μl volume to a final DMSO concentration of 1%. The reaction is stopped by adding 25 μl 1 mM 1,4 dinitrophenylhydrazine in 1N HCl, After a second 10 min incubation at room temperature, 175 μl 0.6 N NaOH is added and the formed hydrazone is detected by measuring the absorbance at 450 nm. All products were purchased from Sigma.

The table herein below, enlists the effect of the compounds according to the invention on the DAAO-activity.

| | H₂O₂ method | keto acid method |
|---|---|---|
| Int. No. | pIC50 | pIC50 |
| 5 | 7.6 | 7.1 |
| 14 | — | 6.1 |
| 4 | 6.1 | 5.9 |
| 8 | 6.1 | 5.9 |
| 2 | 6.0 | 5.9 |
| 12 | — | 5.9 |
| 6 | 5.5 | 5.4 |
| 13 | — | 5.4 |
| 7 | 4.7 | 4.9 |
| 9 | — | 4.7 |
| 15 | — | 4.4 |
| Co. No. | H₂O₂ method | keto acid method |
| 16 | 7.5 | 7.0 |
| 18 | 7.5 | 6.9 |
| 27 | 7.3 | 6.7 |
| 25 | 6.2 | 6.1 |
| 22 | 6.2 | 6.1 |
| 23 | 6.1 | 6.0 |
| 21 | 6.2 | 6.0 |
| 17 | 6.2 | 6.0 |
| 20 | 5.3 | 5.9 |
| 26 | 5.5 | 5.7 |
| 28 | 5.4 | 5.7 |
| 29 | — | 5.4 |
| 19 | 5.4 | 5.3 |
| 15 | — | 5.2 |

### D. Composition Examples

The following formulations exemplify typical pharmaceutical compositions suitable for systemic or topical administration to animal and human subjects in accordance with the present invention.

“Active ingredient” (A.I.) as used throughout these examples relates to a compound of formula (I) or a pharmaceutically acceptable addition salt thereof.

### Example D.1

### Film-Coated Tablets

Preparation of Tablet Core

A mixture of A.I. (100 g), lactose (570 g) and starch (200 g) was mixed well and thereafter humidified with a solution of sodium dodecyl sulfate (5 g) and polyvinyl-pyrrolidone (10 g) in about 200 ml of water. The wet powder mixture was sieved, dried and sieved again. Then there was added microcrystalline cellulose (100 g) and hydrogenated vegetable oil (15 g). The whole was mixed well and compressed into tablets, giving 10,000 tablets, each comprising 10 mg of the active ingredient.

Coating

To a solution of methyl cellulose (10 g) in denaturated ethanol (75 ml) there was added a solution of ethyl cellulose (5 g) in CH₂Cl₂ (150 ml). Then there were added CH₂Cl₂ (75 ml) and 1,2,3-propanetriol (2.5 ml). Polyethylene glycol (10 g) was molten and dissolved in dichloromethane (75 ml). The latter solution was added to the former and then there were added magnesium octadecanoate (2.5 g), polyvinyl-pyrrolidone (5 g) and concentrated color suspension (30 ml) and the whole was homogenated. The tablet cores were coated with the thus obtained mixture in a coating apparatus.

1. The use of a DAAO inhibiting compound for the manufacture of a medicament for the treatment of mental disorders, said compound having the formula 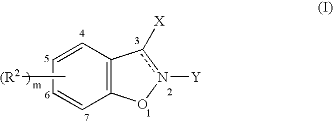
the N-oxide forms, the pharmaceutically acceptable addition salts and the stereochemically isomeric forms thereof, wherein
m represents an integer from 1 to 3;
X represents hydroxy, amino, -oxo or -Z-R¹;
Y is absent or represents —(C═O)—R⁶;
Z represents carbonyl, -oxy-carbonyl-, ═N-carbonyl- or —NR⁵-carbonyl;
R¹ represents hydrogen, C₁₋₄alkyl, C₁₋₄alkyloxy-, Ar¹, Ar²—C₁₋₄alkyl-, —NR³R⁴ or -Het¹;
R² represents hydrogen, halo, hydroxy, nitro, cyano, hydroxycarbonyl-, amino, mono- or di (C₁₋₄alkyl)amino-, C₁₋₆alkyloxycarbonyl-, C₁₋₄alkyloxycarbonylC₁₋₄alkyloxy-, C₁₋₄alkyloxy- optionally substituted with one or more halo atoms or R² represents C₁₋₄alkyl optionally substituted with one or more halogen atoms;
R³ and R⁴ are each independently selected from hydrogen, Het², Ar³, C₁₋₄alkyl or C₁₋₄alkyl substituted with one or more substitutents selected from halo, hydroxy or C₁₋₄alkyloxy-;
R⁵ represents hydrogen, C₁₋₄alkyl, C₁₋₄alkylcarbonyl, C₁₋₄alkyloxycarbonyl- or Ar⁴-carbonyl-;
R⁶ represents a substitutent selected from the group consisting of C₁₋₄alkyl, C₁₋₄alkyloxy-, Ar⁵, Ar⁶—C₁₋₄alkyl-, —NR⁷R⁸ or Het³;
R⁷ and R⁸ are each independently selected from hydrogen, Het⁴, Ar⁷, C₁₋₄alkyl or C₁₋₄alkyl substituted with one or more substitutents selected from halo, hydroxy or C₁₋₄alkyloxy-;
Het¹ represents a heterocycle selected from oxazolyl, isoxazolyl, thiazolyl, isothiazolyl, imidazolyl, pyrazolyl, benzisoxazolyl, benzimidazolyl or benzothiazolyl wherein said heterocycle is optionally substituted with one or more substitutents each independently selected from the group consisting of amino, C₁₋₄alkyl, hydroxy-C₁₋₄alkyl-, phenyl, phenyl-C₁₋₄alkyl- and phenyl substituted with one or more halo substitutents;
Het² represents a heterocycle selected from oxazolyl, isoxazolyl, thiazolyl, isothiazolyl, imidazolyl, pyrazolyl, benzisoxazolyl, benzimidazolyl or benzothiazolyl wherein said heterocycle is optionally substituted with one or more substitutents each independently selected from the group consisting of amino, C₁₋₄alkyl, hydroxy-C₁₋₄alkyl-, phenyl, phenyl-C₁₋₄alkyl- and phenyl substituted with one or more halo substitutents;
Het³ represents a heterocycle selected from oxazolyl, isoxazolyl, thiazolyl, isothiazolyl, imidazolyl, pyrazolyl, benzisoxazolyl, benzimidazolyl or benzothiazolyl wherein said heterocycle is optionally substituted with one or more substitutents each independently selected from the group consisting of amino, C₁₋₄alkyl, hydroxy-C₁₋₄alkyl-, phenyl, phenyl-C₁₋₄alkyl- and phenyl substituted with one or more halo substitutents;
Het⁴ represents a heterocycle selected from oxazolyl, isoxazolyl, thiazolyl, isothiazolyl, imidazolyl, pyrazolyl, benzisoxazolyl, benzimidazolyl or benzothiazolyl wherein said heterocycle is optionally substituted with one or more substitutents each independently selected from the group consisting of amino, C₁₋₄alkyl, hydroxy-C₁₋₄alkyl-, phenyl, phenyl-C₁₋₄alkyl- and phenyl substituted with one or more halo substitutents;
Ar¹, Ar², Ar³, Ar⁴, Ar⁵, Ar⁶ or Ar⁷ each independently represents phenyl optionally substituted one or where possible two or more substitutents selected from halo, nitro, C₁₋₄alkyl, hydroxy or C₁₋₄alkyloxy-.

2. The use according to claim 1 wherein for the compounds of formula (I)
m represents an integer from 1 to 3;
X represents -oxo or -Z-R¹;
Y is absent when X represents -Z-R¹ and —(C═O)—R⁶ when X represents oxo;
Z represents carbonyl, -oxy-carbonyl- or —NR⁵-carbonyl-;
R¹ represents C₁₋₄alkyl, Ar¹, Ar¹—C₁₋₄alkyl-, —NR³R⁴ or -Het¹;
R² represents hydrogen, halo, nitro, hydroxycarbonyl-, C₁₋₄alkyloxy or C₁₋₄alkyl;
R³ and R⁴ are each independently selected from hydrogen, Ar³ or C₁₋₄alkyl;
R⁵ represents hydrogen, C₁₋₄alkylcarbonyl- or Ar⁴-carbonyl-;
R⁶ represents a substitutent selected from the group consisting of C₁₋₄alkyl, Ar⁵ Ar⁶-C₁₋₄alkyl- or NR⁷R⁸;
R⁷ and R⁸ are each independently selected from hydrogen, Het⁴ or C₁₋₄alkyl;
Het¹ represents a heterocycle selected from oxazolyl, isoxazolyl, imidazolyl or pyrazolyl wherein said heterocycle is optionally substituted with one, two or three substitutents selected from the group consisting of amino, C₁₋₄alkyl, hydroxy-C₁₋₄alkyl, phenyl, phenyl-C₁₋₄alkyl- and phenyl substituted with one or more halo substitutents, in particular said heterocycle is substituted with one or more substitutents selected from the group consisting of C₁₋₄alkyl, phenyl or phenyl substituted with one or more halo substitutents; in a particular embodiment Het¹ represents a heterocycle selected from isoxazolyl and pyrazolyl wherein said heterocycle is substituted with one or more substitutents selected from the group consisting of amino, C₁₋₄alkyl, hydroxy-C₁₋₄alkyl, phenyl, phenyl-C₁₋₄alkyl- and phenyl substituted with one or more halo substitutents, in particular said heterocycle is substituted with one or more substitutents selected from the group consisting of C₁₋₄alkyl, phenyl or phenyl substituted with one or more halo substitutents;
Het⁴ represents a heterocycle selected from oxazolyl or isoxazolyl, wherein said heterocycle is optionally substituted with one or more substitutents selected from the group consisting of amino, C₁₋₄alkyl, hydroxy-C₁₋₄alkyl-, phenyl, phenyl-C₁₋₄alkyl and phenyl substituted with one or more halo substitutents, in particular said heterocycle is substituted with one or more substitutents selected from C₁₋₄alkyl, phenyl or phenyl substituted with one or more halo substitutents; in a particular embodiment Het⁴ represents isoxazolyl substituted with one or more substitutents selected from C₁₋₄alkyl, phenyl or phenyl substituted with one or more halo substitutents;
Ar¹, Ar², Ar³, Ar⁴, Ar⁵ or Ar⁶ each independently represents phenyl;

3. A compound of formula (I) a defined in claim 1, provided however that when;
Z is -oxycarbonyl and R¹ is chloro- or nitro-phenyl-, then R² is not methyloxy-, ethyloxy-, chloro or fluoro,
Z is -oxycarbonyl and R¹ is methyl, methyloxy-, ethyloxy-, phenyl, chlorophenyl, nitrophenyl, isoxazolyl substituted with chloro or methyl or when R¹ is pyrazolyl substituted with ethyl and methyl, then R² is not hydrogen, chloro, fluoro, bromo, ethyloxy, methyloxy or methyl,
Z is —NR⁵-carbonyl and R¹ is methyl, methyloxy-, ethyloxy-, t-butyloxy-, benzyloxy-, phenyl or di-chlorophenyl, then R² is not hydrogen, halo, methyl or trifluoromethyl, or
Z is oxycarbonyl and R³ or R⁴ is a methyl, isopropyl, propyl, t-butyl or an isoxazolyl substituted with either chloro, one methyl substitutent or with one methyl and one di-chloro-phenyl substitutent, then R² is not hydrogen, chloro or methyl.

4. A compound of formula (I) wherein R¹ is a heterocycle Het¹ selected from the group consisting of isoxazolyl, pyrazolyl or benzisoxazolyl wherein said Het¹ is optionally substituted with one or more substitutents each independently selected from the group consisting of C₁₋₄alkyl, phenyl and phenyl substituted with one or more halo substitutents,
provided that when R¹ is a substituted isoxazolyl or a substituted pyrazolyl, then R² is not hydrogen, chloro or methyl.

5. A compound of formula (I) as claimed in claim 3 4, for use as a medicine.

6. Use of a compound of formula (I) as claimed in claim 1 in the manufacture of a medicament for the treatment of schizophrenia.

7. A method of treating a mental disorder such as schizophrenia, the method comprising administering to an animal in need of such treatment a therapeutically effective amount of a compound of formula (I).

8. The use of intermediates with DAAO inhibiting activity in the manufacture of a medicament for treatment of mental disorders, said intermediates having formula (Ia) or (Ig) 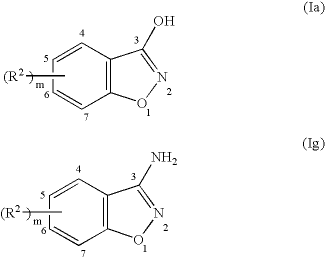
the N-oxide forms, the pharmaceutically acceptable addition salts and the stereochemically isomeric forms thereof, wherein
m represents an integer from 1 to 3;
R² represents hydrogen, halo, hydroxy, nitro, cyano, hydroxycarbonyl-, amino, mono- or di (C₁₋₄alkyl)amino-, C₁₋₆alkyloxycarbonyl-, C₁₋₄alkyloxycarbonylC₁₋₄alkyloxy-, C₁₋₄alkyloxy- optionally substituted with one or more halo atoms or R² represents C₁₋₄alkyl optionally substituted with one or more halogen atoms.

9. A compound of formula 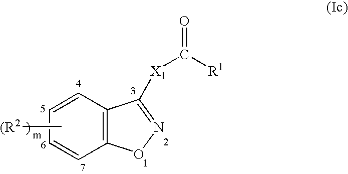
the N-oxide forms, the pharmaceutically acceptable addition salts and the stereochemically isomeric forms thereof, wherein
m represents an integer from 1 to 3;
X₁ represents O or NR⁵;
R¹ represents C₁₋₄alkyl, C₁₋₄alkyloxy-, Ar¹, Ar²—C₁₋₄alkyl-, —NR³R⁴ or Het¹;
R² represents hydrogen, halo, hydroxy, nitro, hydroxycarbonyl-, amino, mono- or di (C₁₋₁₄alkyl)amino, C₁₋₁₆alkyloxycarbonyl-, C₁₋₄alkyloxycarbonylC₁₋₄alkyloxy-, C₁₋₁₄alkyloxy- optionally substituted with one or more halo atoms or R² represents C₁₋₄alkyl optionally substituted with one or more halogen atoms;
R³ and R⁴ are each independently selected from hydrogen, Het², phenyl, C₁₋₄alkyl or C₁₋₄alkyl substituted with one or more substitutents selected from halo, hydroxyl, phenyl or C₁₋₄alkyloxy-;
R⁵ represents hydrogen, C₁₋₄alkyl, phenyl-carbonyl- or C₁₋₄alkyl-carbonyl-;
Het¹ represents a heterocycle selected from oxazolyl, isoxazolyl, thiazolyl, isothiazolyl, imidazolyl, pyrazolyl, benzisoxazolyl, benzimidazolyl or benzothiazolyl wherein said heterocycle is optionally substituted with one or more substitutents each independently selected from the group consisting of amino, C₁₋₄alkyl, hydroxy-C₁₋₄alkyl-, phenyl, phenyl-C₁₋₄alkyl- and phenyl substituted with one or more halo substitutents;
Het² represents a heterocycle selected from oxazolyl, isoxazolyl, thiazolyl, isothiazolyl, imidazolyl, pyrazolyl, benzisoxazolyl, benzimidazolyl or benzothiazolyl wherein said heterocycle is optionally substituted with one or more substitutents each independently selected from the group consisting of amino, C₁₋₄alkyl, hydroxy-C₁₋₄alkyl-, phenyl, phenyl-C₁₋₄alkyl- and phenyl substituted with one or more halo substitutents,
provided that when;
X₁ is —O— and R¹ is methyl, methyloxy-, ethyloxy-, phenyl, chlorophenyl, nitrophenyl, isoxazolyl substituted with chloro or methyl or when R¹ is pyrazolyl substituted with ethyl and methyl, then R² is not hydrogen, chloro, fluoro, bromo or methyl,
X₁ is NR⁵ and R¹ is methyl, methyloxy-, ethyloxy-, t-butyloxy-, benzyloxy-, phenyl or di-chloro-phenyl, then R² is not hydrogen, halo, methyl or trifluoromethyl,
X₁ is —O— and R³ or R⁴ is a methyl, isopropyl, propyl, t-butyl or an isoxazolyl substituted with either chloro, one methyl substitutent or with one methyl and one di-chloro-phenyl substitutent, then R² is not hydrogen, chloro or methyl.

10. A compound according to claim 9 wherein
m is 1;
X₁ represents O or NR⁵;
R¹ is NR³R⁴ or Het¹;
R² is hydrogen, halo or R² represents C₁₋₄alkyl;
R³ and R⁴ are each independently selected from hydrogen, Het² and C₁₋₄alkyl;
R⁵ represents hydrogen or C₁₋₄alkyl-carbonyl-;
Het¹ is isoxazolyl or imidazolyl each independently substituted with one or more substitutents selected from C₁₋₄alkyl and phenyl substituted with one or more halo substitutents;
Het² is isoxazolyl substituted with one or more substitutents selected from C₁₋₄alkyl and phenyl substituted with one or more halo substitutents.

11. A compound according to claim 9 wherein
m is 1;
X₁ represents NR⁵;
R¹ is NR³R⁴ or Het¹;
R² is hydrogen, chloro or methyl;
R³ represents hydrogen and R⁴ is C₁₋₄alkyl, phenyl or C₁₋₄alkyl substituted with phenyl;
R⁵ represents hydrogen, phenyl-carbonyl- or C₁₋₄alkyl-carbonyl-;
Het¹ is isoxazolyl or imidazolyl each independently substituted with one or more substitutents selected from C₁₋₄alkyl and phenyl substituted with one or more halo substitutents;
Het² is isoxazolyl substituted with one or more substitutents selected from C₁₋₄alkyl and phenyl substituted with one or more halo substitutents.

12. A compound according to claim 9 wherein X₁ represents O and R³ and R⁴ are each independently selected from Het², Ar³, C₁₋₄alkyl or C₁₋₄alkyl substituted with one or more substitutents selected from halo, hydroxy or C₁₋₄alkyloxy-.

13. (canceled)

14. (canceled)

15. A pharmaceutical composition comprising a pharmaceutically acceptable carrier and, as active ingredient, an effective DAAO inhibitory amount of a compound as described in claim 1.

16. A method of treating a mental disorder such as schizophrenia, the method comprising administering to an animal in need of such treatment a therapeutically effective amount of a compound of formula (I).

17. A method of treating a mental disorder such as schizophrenia, the method comprising administering to an animal in need of such treatment a therapeutically effective amount of an intermediate of formula (Ia) or (Ig).

18. A compound of formula 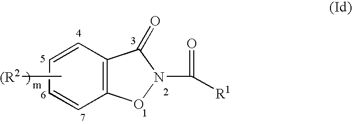
the N-oxide forms, the pharmaceutically acceptable addition salts and the stereochemically isomeric forms thereof, wherein
m represents an integer from 0 to 3;
R¹ represents hydrogen, C₁₋₄alkyl, C₁₋₄alkyloxy, Ar¹, Ar²—C₁₋₄alkyl, NR³R⁴ or Het¹;
R² represents hydrogen, halo, hydroxy, nitro, cyano, hydroxycarbonyl-, amino, mono- or di (C₁₋₄alkyl)amino, C₁₋₆alkyloxycarbonyl-, C₁₋₄alkyloxycarbonylC₁₋₄alkyloxy-, C₁₋₄alkyloxy- optionally substituted with one or more halo atoms or R² represents C₁₋₄alkyl- optionally substituted with one or more halogen atoms;
R³ and R⁴ are each independently selected from hydrogen, Het², Ar³, C₁₋₄alkyl or C₁₋₄alkyl substituted with one or more substitutents selected from halo, hydroxy or C₁₋₄alkyloxy-;
Het¹ represents a heterocycle selected from oxazolyl, isoxazolyl, thiazolyl, isothiazolyl, imidazolyl, pyrazolyl, benzisoxazolyl, benzimidazolyl or benzothiazolyl wherein said Het¹ is optionally substituted with one or more substitutents each independently selected from the group consisting of amino, C₁₋₄alkyl, hydroxy-C₁₋₄alkyl-, phenyl, phenyl-C₁₋₄alkyl- and phenyl substituted with one or more halo substitutents;
Het² represents a heterocycle selected from oxazolyl, isoxazolyl, thiazolyl, isothiazolyl, imidazolyl, pyrazolyl, benzisoxazolyl, benzimidazolyl or benzothiazolyl wherein said Het¹ is optionally substituted with one or more substitutents each independently selected from the group consisting of amino, C₁₋₄alkyl, hydroxy-C₁₋₄alkyl-, phenyl, phenyl-C₁₋₄alkyl- and phenyl substituted with one or more halo substitutents;
Ar¹, Ar² or Ar³ each independently represents phenyl optionally substituted one or where possible two or more substitutents selected from halo, nitro, C₁₋₄alkyl, hydroxy or C₁₋₄alkyloxy.
provided that when;
m represents 1 and R¹ represents chloro- or nitro-phenyl, then R² is not hydrogen, methoxy, ethoxy, chloro or fluoro;
R¹ represents ethoxy or methoxy, then R² is not hydrogen, bromo, fluoro or chloro;
R¹ represents methyl, then R² is not hydrogen, bromo or chloro.

19. (canceled)

20. (canceled)

21. A method of treating a mental disorder such as schizophrenia, the method comprising administering to an animal in need of such treatment a therapeutically effective amount of an intermediate of formula (Id).
